(19) United States
(12) Patent Application Publication
(10) Pub. No.: US 2025/0235125 A1
(43) Pub. Date: Jul. 24, 2025

(54) CROSS-PERIOD BRAIN FINGERPRINT IDENTIFICATION METHOD WITH PARADIGM ADAPTIVE DECOUPLING AND SYSTEM THEREOF

(71) Applicant: Hangzhou Dianzi University, Hangzhou City (CN)

(72) Inventors: Wanzeng KONG, Hangzhou City (CN); Honggang LIU, Hangzhou City (CN); Dongjun LIU, Hangzhou City (CN); Xuanyu JIN, Hangzhou City (CN); Li ZHU, Hangzhou City (CN)

(21) Appl. No.: 18/922,777

(22) Filed: Oct. 22, 2024

(30) Foreign Application Priority Data
Jan. 22, 2024 (CN) 202410087933.2

(51) Int. Cl. A61B 5/117; A61B 5/00; A61B 5/31; A61B 5/369; H04L 9/40

(52) U.S. Cl. CPC A61B 5/117; A61B 5/31; A61B 5/369; A61B 5/7267; H04L 63/0861

(57) ABSTRACT

Provided is a cross-period brain fingerprint identification method with paradigm adaptive decoupling and a system thereof. The method includes: extracting a feature representation from original electroencephalogram data by a feature extractor; effectively separating identity-related features and paradigm task-related features from highly coupled electroencephalogram information; and further learning domain invariant features with identity identification ability through domain adversarial training. According to the method, three decouplers are introduced to perform feature decoupling on features extracted by a feature extraction module. At the same time, three classifiers are introduced to pass through a domain label, an identity label and a paradigm task label, and the decouplers are guided to decouple paradigm task features and identity features effectively through adversarial training.

### CROSS-REFERENCE TO RELATED APPLICATION

This patent application claims the benefit and priority of Chinese Patent Application No. 202410087933.2 filed with the China National Intellectual Property Administration on Jan. 22, 2024, the disclosure of which is incorporated by reference herein in its entirety as part of the present application.

### TECHNICAL FIELD

The present disclosure belongs to the technical field of biometric identification based on electroencephalogram signals, and in particular relates to a cross-period brain fingerprint identification method with paradigm adaptive decoupling and a system thereof.

### BACKGROUND

As a highly identifiable physiological feature, an electroencephalogram signal has great potential in the field of biometric identification. With the continuous development and popularization of consumer wearable devices, users can acquire electroencephalogram data through sensors in the devices to realize contactless identity identification. Compared with traditional biometric technologies (such as face identification and fingerprint identification), the electroencephalogram signal identification is more confidential, more difficult to be stolen, and revocable and revisable. On the other hand, compared with conventional biological features, the electroencephalogram signal identification has obvious advantages in some specific scenarios, such as some highly confidential and safe occasions, or in some new technology usage scenarios, such as virtual reality (VR) and augmented reality (AR).

However, different methods of collecting electroencephalogram signals lead to different features and manifestations of electroencephalogram signals, which challenges the generalization and identification ability of the model. In addition, electroencephalogram signals are very sensitive to internal and external noises, such as physiological state, psychological state and environmental noise, which makes it difficult to guarantee the stability and reliability of the model in different periods. Therefore, most of the work is to collect electroencephalogram signals in a single paradigm or in a single period, which is not consistent with the actual application scenario.

In order to overcome these challenges, the present disclosure provides a cross-period brain fingerprint identification method with paradigm adaptive decoupling. The method can effectively separate identity-related features and paradigm task-related features from highly coupled electroencephalogram information; and further learn domain invariant features with identity identification ability through domain adversarial training. The present disclosure can be adaptive to electroencephalogram signals collected by various paradigms under a cross-period condition, which has a high practical value in practical application scenarios and provides innovative solutions for the fields of biometric identification, identity verification and the like.

### SUMMARY

According to the shortcomings of the prior art, the present disclosure provides a cross-period brain fingerprint identification method with paradigm adaptive decoupling and a system thereof. First, a feature extractor extracts is a feature representation from original electroencephalogram data; thereafter, effectively separates identity-related features and paradigm task-related features from highly coupled electroencephalogram information; and finally, learns domain invariant features with identity identification ability through domain adversarial training.

In a first aspect, the present disclosure provides a cross-period brain fingerprint identification method with paradigm adaptive decoupling, including the following steps:
Step 1: collecting electroencephalogram data
collecting electroencephalogram data of a plurality of subjects at different periods;
Step 2: preprocessing the electroencephalogram data, labeling the electroencephalogram data with identity labels of subjects, and then dividing the electroencephalogram data into a source domain and a target domain according to a sequence of collecting periods, wherein the source domain is a data set with known identity labels of the subjects, and the target domain is a data set with identity labels to be predicted;
Step 3: constructing a feature extraction module with paradigm adaptive decoupling, and training and testing the feature extraction module;
the feature extraction module with paradigm adaptive decoupling includes a multi-scale convolution module, a graph convolution module and an attention embedding module;
the multi-scale convolution module includes a plurality of parallel one-dimensional convolution layers, a splicing layer, a fusion layer and a filtering layer; the plurality of one-dimensional convolution layers have convolution kernels with different sizes; the multi-scale convolution module receives data of the source domain and the target domain, processes the data in multi-time dimension through a plurality of parallel one-dimensional convolution kernels with different sizes, and outputs features of different levels as an input of the splicing layer; the splicing layer splices an output of one-dimensional convolution layers of different levels as an input of the fusion layer; the fusion layer fuses the features learned by different convolution kernels output by the splicing layer, and flattens the features as an input of the filtering layer;
the graph convolution module includes a plurality of parallel graph convolution networks, and is configured to mine a topological relation and spatial information between channels in a data-driven manner;
the attention embedding module is configured to act on outputs of graph convolution of different levels, and transform a graph structure into an embedding vector through an attention mechanism as an input of decouplers;

Step 4:, constructing a decoupling module for decoupling features and respective classifiers, and training and testing the decoupling module and the classifiers;
wherein the decoupling module for decoupling features includes an intra-domain specific identity information decoupler, an inter-domain invariant identity information decoupler, a paradigm task information decoupler, a first mutual information network and a second mutual information network;
the intra-domain specific identity information decoupler is configured to decouple a feature representation hₒᵤₜ extracted by the feature extraction module into a domain specific identity feature representation h_{sped-id};
the inter-domain invariant identity information decoupler is configured to decouple the feature representation hₒᵤₜ extracted by the feature extraction module into a domain invariant identity feature representation h_{inv-id};
the paradigm task information decoupler is configured to decouple the feature representation hₒᵤₜ extracted by the feature extraction module into a paradigm task-related feature representation hₜₐₛₖ;
the first mutual information network is configured to calculate a first mutual information loss between the paradigm task-related feature representation hₜₐₛₖ and the domain invariant identity feature representation h_{inv-id}; the second mutual information network is configured to calculate a second mutual information loss between the domain specific identity feature representation h_{sped-id} and the domain invariant identity feature representation h_{inv-id};
network parameters of the decoupling module are updated through the first mutual information loss and the second mutual information loss, so as to reduce the mutual information between decoupled features and obtain a better decoupling effect;
the intra-domain specific identity information decoupler, the inter-domain invariant identity information decoupler, the paradigm task information decoupler, the first mutual information network and the second mutual information network all consist of fully connected layer networks, and the three types of decoupled feature representations, namely hₜₐₛₖ, h_{inv-id} and h_{sped-id}, will be used as the input of each classifier, the mutual information networks aim to calculate mutual information loss, and network parameters are updated through the loss, so as to reduce the mutual information between decoupled features and obtain a better feature decoupling effect;
the classifiers include a domain classifier, an identity classifier and a paradigm task classifier;
the domain classifier performs domain adversarial training to reduce distribution differences between different domains and obtain domain invariant features; and takes the domain invariant identity feature representation h_{inv-id} as the input, which consists of a fully connected layer and a Softmax activation function;
the identity classifier aims at obtaining accurate classification information; takes the domain specific identity feature representation h_{sped-id} and the domain invariant identity feature representation h_{inv-id} as the input, which consists of a fully connected layer and a Softmax activation function;
the paradigm task classifier aims at promoting a decoupling effect and reducing the influence of interference information on an identity identification task; takes the paradigm task-related feature representation hₜₐₛₖ as the input, which consists of a fully connected layer and a Softmax activation function; and
Step 5: using the feature extraction module, the decoupling module and the classifiers which have been trained and verified to realize cross-period brain fingerprint identification.

Preferably, in Step 2, preprocessing the electroencephalogram data includes: filtering and down-sampling the electroencephalogram data collected in Step 1, and then fragmenting the electroencephalogram data to obtain a plurality of fragments with a sample length of L.

Preferably, in Step 2, dividing the electroencephalogram data into the source domain and the target domain according to the sequence of collecting periods means that: in a time sequence, the period data collected first is taken as the source domain with identity labels which is denoted as [symbol] _{S}={X_{S}, Y_{S}}={(x_{S}¹,y_{S}¹), ⋅ ⋅ ⋅ , (x_{S}ⁿ^{_{S}},y_{S}^{N}^{_{S}})}, n_{S} represents the number of samples in the source domain, x_{S}ⁱ|ᵢ₌₁ⁿ^{_{S}}∈ [symbol] ^{d×L} represents an electroencephalogram data fragment in a period of time, yₛⁱ|ᵢ₌₁ⁿ^{_{S}}∈ [symbol] ^{C} represents an identity label of an i-th subject in the source domain, and C represents the number of the subjects; the period data collected later is taken as unlabeled target domain of identities to be predicted, which is denoted as [symbol] _{T}={X_{T}}={x_{T}¹, ⋅ ⋅ ⋅ , x_{T}ⁿ^{ₜ}}, x_{T}ⁱ|ᵢ₌₁ⁿ^{ₜ}∈ [symbol] ^{d×L} represents an electroencephalogram data fragment in a period of time, and nₜ represents the number of samples in the target domain.

Preferably, in Step 3, the kernel size of each type of one-dimensional convolution layer is determined by the proportion coefficient aₖ|ₖ₌₁^{K}∈ [symbol] and the sample length L, and the proportion coefficient is artificially set, where k represents the convolution kernel of a k-th type of one-dimensional convolution layers.

In the present disclosure, aₖ=[0.1, 0.2, 0.5], that is, there are three types of one-dimensional convolution kernels with different sizes.

The scale of the time convolution kernel of the k-th type is denoted as [symbol] ^{k} and defined as:

$${𝒜}^{k} = ( 1 , {α}_{k} \cdot L ) \qquad ( 1 )$$

Preferably, in Step 3, the multi-scale one-dimensional convolution layer is specifically expressed as:

$${X}_{T \_ out}^{k} = {Φ}_{log} ( {ℱ}_{AP} ( {Φ}_{square} ( {ℱ}_{Conv 2 D} ( {X}_{i} , {S}_{T}^{k} ) ) ) ) \qquad ( 2 )$$

wherein Xᵢ∈ [symbol] ^{C×L}˜ [symbol] _{S/T},i∈[1, ⋅ ⋅ ⋅ ,N] represents the input of the multi-scale one-dimensional convolution layer, that is, the preprocessed electroencephalogram data, N is the number of sample fragments of the preprocessed electroencephalogram data, C is the number of channels of the electroencephalogram collecting device, and L is the length of the set electroencephalogram data in the time dimension; X_{T_out}^{k}∈ [symbol] ^{t×C×f}^{ₖ} represents the output of the multi-scale convolution module, t represents the number of one-dimensional convolution layers, and fₖ represents the feature length of the output features of the k-th type of one-dimensional convolution layers; [symbol] _{Conv2D}(Xᵢ,S_{T}^{k}) represents the convolution operation of Xᵢ using a time convolution kernel with a size of S_{T}^{k}; Φ_{square}(·) is a square function; [symbol] _{AP}(·) represents the average pooling operation, and Φ_{log}(·) represents a logarithmic function.

The splicing layer is specifically expressed as:

$${X}_{cat}^{i} = Γ ( {X}_{T \_ out}^{1} , … , {X}_{T \_ out}^{K} ) \qquad ( 3 )$$

where Γ(·) represents the serial operation along the feature dimension; X_{cat}ⁱ∈ [symbol] ^{t×C×Σf}^{ₖ} will be used as the input of the fusion layer.

The fusion layer uses a 1*1 convolution layer to fuse the features learned by different convolution kernels; the number of convolution kernels in the 1*1 convolution layer is set to t. Preferably, Leaky-ReLU is used as the activation function, and the average pooling layer is used to down-sample the learned representation. After batch standardization, the fused representation from different 1*1 convolution kernels will be flattened and become the attribute of each channel node in the graph representation; therefore, the attention fusion representation X̅ⁱ_{fuse} for each X_{cat}ⁱ can be calculated as:

$${{\overline{X}}^{i}}_{fuse} = {ℱ}_{bn} ( {ℱ}_{AP} ( {Φ}_{L - ReLU} ( {ℱ}_{fuse} ( {ℱ}_{dropout} ( {ℱ}_{bn} ( {X}_{cat}^{i} ) ) ) ) ) ) \qquad ( 4 )$$

where [symbol] _{bn}(·) is a batch normalization function, [symbol] _{dropout}(·) is a random dropout layer preventing over-fitting, [symbol] _{fuse}(·) is a 1*1 convolution function, Φ_{L-ReLU}(·) is a Leaky-ReLU activation function; [symbol] _{AP}(·) represents the average pooling operation.

X̅ⁱ_{fuse} is reshaped as Xⁱ_{fuse}∈ [symbol] ^{C×t* 0.5*Σf}^{ₖ}:

$${X}_{fuse}^{i} = {ℱ}_{reshape} ( {{\overline{X}}^{i}}_{fuse} ) \qquad ( 5 )$$

The graph convolution module includes a plurality of graph convolution layers connected in series. Specifically, a messaging framework is used to realize the graph convolution layer (GNN):

$${X}_{out} = GNN ( A , {X}_{fuse}^{i} ) \qquad ( 6 )$$

where A=Xⁱ_{fuse}Xⁱ_{fuse}^{T}∈ [symbol] ^{d×d}, d represents the number of channels of electroencephalogram features;

The attention embedding module obtains the embedded vector representation of the features through the attention mechanism. Specifically, first, the global mean representation

$${h}_{mean} = \frac{1}{d} {\sum }_{i = 1}^{d} {x}_{out}^{i}$$

is calculated, and then each channel xₒᵤₜⁱ|ᵢ^{d} and hₘₑₐₙ are subjected to inner products to obtain a similarity representation, which can be regarded as the expression of the importance degree belonging to each channel. Finally, the final embedded vector representation is obtained by weighted summation:

$${h}_{out} = soft max ( {h}_{mean} {X}_{out}^{T} ) {X}_{out} \qquad ( 8 )$$

Preferably, in the training process, the decouplers use adversarial training to achieve feature decoupling, specifically, the intra-domain specific identity information decoupler, the inter-domain invariant identity information decoupler and the paradigm task information decoupler decouple hₒᵤₜ into the domain specific identity feature representation h_{sped-id}, the domain invariant identity feature representation h_{inv-id} and the paradigm task-related feature representation hₜₐₛₖ, respectively.

Preferably, the intra-domain specific identity feature h_{sped-id} and the inter-domain invariant identity feature h_{inv-id} are constrained by L1 norm.

Thereafter, the identity classifier [symbol] _{c-id}(·) and the paradigm task classifier [symbol] _{c-task} are trained to realize correct classification, which are iteratively optimized through a cross entropy loss.

$${ℒ}_{c 1} = - {𝔼}_{( {x}_{s} , {y}_{s} ) ∼ {\tilde{𝒟}}_{s}} \sum {y}_{s} log ( {ℱ}_{C - id} ( {h}_{spec - id} / {h}_{inv - id} ) ) \qquad ( 9 )$$

$${ℒ}_{c 2} = - {𝔼}_{x ∼ {\tilde{𝒟}}_{s / t}} \sum {y}_{task} log ( {ℱ}_{C - task} ( {h}_{task} ) ) \qquad ( 10 )$$

where [symbol] ₍ₓ_{_{S}}_{,y}_{_{S}}_{)˜\tilde{D}}_{_{S}}, represents a feature representation extracted from the source domain data; [symbol] _{x˜\tilde{D}}_{_{s/t}} represents a feature representation extracted from the source domain data or the target domain data; y_{S} represents a real identity label of the source domain data; yₜₐₛₖ represents the task label that the subject carries out when electroencephalogram data is collected○

In the training process, the domain classifier [symbol] _{C-domain}(·) realizes the distribution alignment of the source domain and the target domain in a domain adversarial manner, specifically, in an identity-related feature subspace, the source domain and the target domain are aligned through domain adversarial training:

$${ℒ}_{d} = - {𝔼}_{x ∼ {\tilde{𝒟}}_{S}} [ log ( {ℱ}_{C - domain} ( {h}_{inv - id} ) ) ] - {{𝔼}_{x ∼ \tilde{𝒟}}}_{t} [ log ( 1 - {ℱ}_{C - domain} ( {h}_{inv - id} ) ) ] \qquad ( 12 )$$

where [symbol] _{x˜\tilde{D}}_{_{S}}[log( [symbol] _{C-domain}(h_{inv-id})] represents that an encouragement that the domain classifier [symbol] _{C-domain}(·) correctly predicts the source domain data, and [symbol] _{x˜\tilde{D}}_{_{S}}[log(1− [symbol] _{C-domain}(h_{inv-id})] represents an encouragement that the features decoupled by the inter-domain invariant identity information decoupler deceive [symbol] _{C-domain}(·) to obtain the domain invariant identity feature.

Preferably, a better decoupling effect is obtained by updating the network through the mutual information loss. That is, the first mutual information loss [symbol] ₁(h_{inv-id},hₜₐₛₖ) between the domain invariant identity feature representation h_{inv-id} and the paradigm task-related feature hₜₐₛₖ is calculated through the first mutual information network, and the second mutual information loss [symbol] ₂ (h_{inv-id}, h_{spec-id}) between the domain invariant identity feature representation h_{inv-id} and the domain specific identity feature representation h_{sped-id} is calculated through the second mutual information network, which are expressed as:

$${ℳℐ}_{1} ( {h}_{inv - id} , {h}_{task} ) = {ℐ}_{1} ( {h}_{inv - id} , {h}_{task} ; θ ) - log ( {e}^{{ℐ}_{1} ( {h}_{inv - id} , {{h}_{task}}^{′} ; θ )} ) \qquad ( 14 )$$

$${ℳℐ}_{2} ( {h}_{inv - id} , {h}_{spec - id} ) = {ℐ}_{2} ( {h}_{inv - id} , {h}_{spec - id} ; θ ) - log ( {e}^{{ℐ}_{2} ( {h}_{inv - id} , {{h}_{spec - id}}^{′} ; θ )} ) \qquad ( 15 )$$

where hₜₐₛₖ and h_{spec-id} represent the edge distributions sampled from hₜₐₛₖ and h_{sped-id}, respectively; [symbol] _{1/2} (·) represents the first mutual information network and the second mutual information network, respectively; θ is a learnable parameter.

In a second aspect, the present disclosure provides a cross-period brain fingerprint identification system, including:
a data collecting module, configured to collect electroencephalogram data;
a data preprocessing module, configured to preprocess the electroencephalogram data;
an identifying module, configured to realize cross-period brain fingerprint identification according to preprocessed electroencephalogram data by using the feature extraction module, the decoupling module and the classifiers which have been trained and verified in advance.

In a third aspect, the present disclosure provides a computer-readable storage medium having a computer program stored thereon, which, when executed in a computer, causes the computer to execute the method.

In a fourth aspect, the present disclosure provides a computing device, including a memory and a processor, wherein executable codes are stored in the memory, and the processor, when executing the executable codes, implement the method.

Through these devices and apparatuses, the present disclosure realizes an efficient and accurate cross-period brain fingerprint identification solution, and brings an innovative technical support for the fields of biometric identification, identity verification and the like.

The present disclosure has the following features and beneficial effects.
1. The present disclosure introduces an intra-domain specific identity information decoupler, an inter-domain invariant identity information decoupler and a paradigm task information decoupler to decouple the features extracted by the feature extraction module.
2. The present disclosure introduces a mutual information loss. That is, the mutual information between the intra-domain specific identity information, the inter-domain invariant identity information and the paradigm task information is reduced through the first mutual information network and the second mutual information network, and the decoupling effect of the network is enhanced.
3. The present disclosure introduces a domain classifier, an identity classifier and a paradigm task classifier to pass through a domain label, an identity label and a paradigm task label, and the decouplers are guided to decouple paradigm task features and identity features effectively through adversarial training.
4. The solution disclosed in the present disclosure is adaptive to the electroencephalogram data collected by various paradigms: the method of the present disclosure can decouple the identity features of the electroencephalogram data collected by various paradigms in the training process, which makes it more in line with the practical application scenarios and improves the practicability.
5. The solution disclosed in the present disclosure has high confidentiality: since electroencephalogram signals are difficult to be stolen, the cross-period brain fingerprint identification technology of the present disclosure has high confidentiality and is expected to become a safe identity verification and identification method.
6. The solution disclosed in the present disclosure has a wide application prospect: the cross-period brain fingerprint identification method of the present disclosure can be applied to the fields of biometric identification, identity verification, security authentication and the like, and provide an innovative technical support for related industries.

### BRIEF DESCRIPTION OF THE DRAWINGS

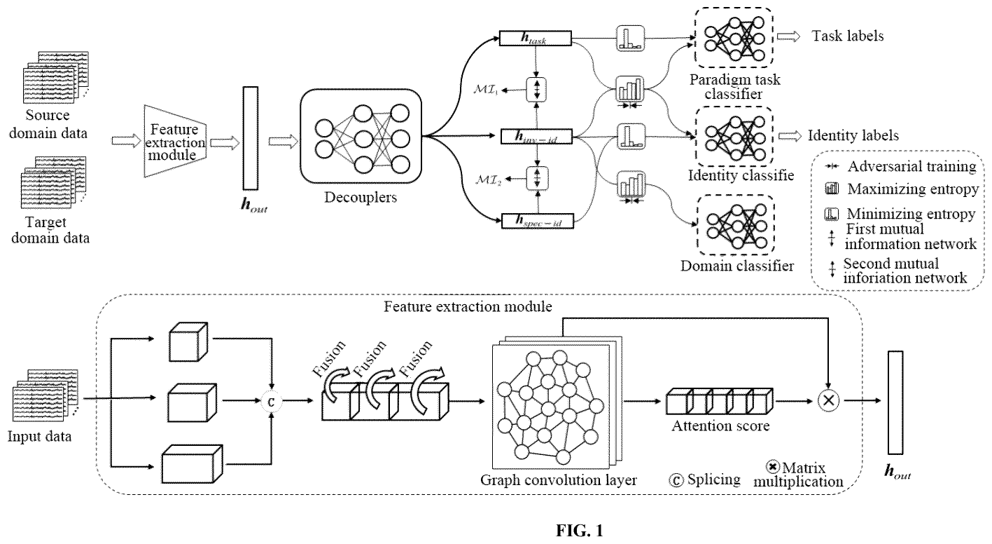

In order to more clearly explain the technical scheme in the embodiment of the present disclosure or the prior art, the drawing required in the description of the embodiment or the prior art will be briefly introduced hereinafter. Obviously, the drawing in the following description only represents some embodiments of the present disclosure. For those skilled in the art, other related drawings can be obtained according to the drawing without creative labor. FIG. 1 shows a working flow chart of a brain fingerprint identification method according to the present disclosure.

### DETAILED DESCRIPTION OF THE EMBODIMENTS

In the case of no contradiction, the embodiments in the present disclosure and the features in the embodiments can be combined with each other. The specific implementation steps of the cross-period brain fingerprint identification method with paradigm adaptive decoupling of the present disclosure are as follows.
1. Electroencephalogram data is collected: electrodes are placed on a scalp by a special device, and electroencephalogram signals of a plurality of subjects are collected at different periods in a non-invasive manner.
2. The electroencephalogram data is preprocessed, labelled with identity labels of subjects, and then divided into a source domain and a target domain according to a sequence of collecting periods, wherein the source domain is a data set with known identity labels of the subjects, and the target domain is a data set with identity labels to be predicted.
2-1: the electroencephalogram data collected in Step 1 is filtered and down-sampled to reduce noise and improve signal quality.
2-2: electroencephalogram features obtained in Step 2-1 are divided to obtain a plurality of fragments with a sample length of L, and corresponding fragments are labelled with an identity label of a subject. The plurality of fragments are divided into a source domain and a target domain according to the sequence of collecting periods. In a time sequence, period data collected first is taken as the source domain with identity labels which is denoted as [symbol] _{S}={X_{S}, Y_{S}}={(x_{S}¹,y_{S}¹), ⋅ ⋅ ⋅ , (x_{S}ⁿ^{_{S}},y_{S}^{N}^{_{S}})}, n_{S} represents the number of samples in the source domain, x_{S}ⁱ|ᵢ₌₁ⁿ^{_{S}}∈ [symbol] ^{d×L} represents an electroencephalogram data fragment in a period of time, yₛⁱ|ᵢ₌₁ⁿ^{_{S}}∈ [symbol] ^{C} represents an identity label of an i-th subject in the source domain, and C represents the number of the subjects. Period data collected later is taken as unlabeled target domain, which is denoted as [symbol] _{T}={X_{T}}={x_{T}¹, ⋅ ⋅ ⋅ , x_{T}ⁿ^{ₜ}}, x_{T}ⁱ|ᵢ₌₁ⁿ^{ₜ}∈ [symbol] ^{d×L} represents an electroencephalogram data fragment in a period of time, and n, represents the number of samples in the target domain.

3. A feature extraction module with paradigm adaptive decoupling is constructed: the feature extraction module with paradigm adaptive decoupling mainly consists of a multi-scale convolution module, a graph convolution module and an attention embedding module. The multi-scale convolution module includes more than one one-dimensional convolution layer, which has convolution kernels with different sizes. The multi-scale convolution module further includes a splicing layer and a fusion layer. The graph convolution module consists of a plurality of parallel graph convolution networks. The attention embedding module acts on an output of graph convolution of different levels, and transforms a graph structure into an embedding vector through an attention mechanism.

4. A decoupling module for decoupling features and respective classifiers thereof are constructed: wherein the decoupling module consists of a plurality of decouplers and mutual information networks. The decouplers includes an intra-domain specific identity information decoupler, an inter-domain invariant identity information decoupler and a paradigm task information decoupler. The decouplers decouple extracted feature representation into domain specific identity feature representation, domain invariant identity feature representation and paradigm task-related feature representation. Both the decouplers and the mutual information networks all consist of fully connected layer networks, and outputs of the decouplers are used as inputs of the classifiers. The mutual information networks aim at reducing mutual information between features and promote better decoupling. The classifiers includes: a domain classifier, which is configured to perform domain adversarial training to reduce distribution differences between different domains and obtain domain invariant features; an identity classifier, which aims at obtaining accurate classification information; a paradigm task classifier, which aims at promoting decoupling effect and reducing influence of interference information on an identity identification task. Each classifier takes the output of the respective decoupler as an input, which consists of a fully connected layer and a Softmax activation function.

5. The network model is trained: the classifiers are trained by using decoupled domain invariant identity information, and model parameters are constantly updated through an optimization algorithm, so that the model can obtain better identification performance on training data.

Through above specific implementation steps, the present disclosure realizes a cross-period brain fingerprint identification method with paradigm adaptive decoupling, which can effectively improve the accuracy and robustness of brain fingerprint identification in practical application scenarios.

Specifically, in Step 1, electroencephalogram cap leads are connected to corresponding brain region of a subject to collect electroencephalogram data.

In Step 2, the electroencephalogram data collected in Step 1 is preprocessed. Original electroencephalogram signal contains frequencies of noises. In order to remove power frequency interference resulted from electroencephalogram collecting device and electromyogram interference of the subject, the electroencephalogram data is down-sampled to 200 Hz, and the original electroencephalogram data is filtered by a Butterworth filter at 1 to 75 Hz.

Obtained electroencephalogram features are divided into fragments with a predetermined time window size of L. The specific window size of the present disclosure is 5 s, and the corresponding fragments are labeled with a label of a subject. The fragments are divided into a source domain and a target domain according to the sequence of collecting periods. The period data collected first is taken as the source domain with identity labels, and the period data collected later is taken as the target domain of the identity labels to be predicted.

According to the further setting of the present disclosure, in Step 3, a feature extraction module with paradigm adaptive decoupling is included. Specifically, the feature extraction module mainly consists of a multi-scale convolution module, a graph convolution module and an attention embedding module. The multi-scale convolution module includes more than one one-dimensional convolution layer, which has convolution kernels with different sizes. The multi-scale convolution module further includes a splicing layer, a fusion layer and a transition layer. A size of a one-dimensional convolution kernel is determined according to different proportions of the sample duration L, and the proportion coefficient is denoted as aₖ|ₖ₌₁^{K}∈ [symbol] , where k represents a convolution kernel of a k-th type of time convolution layer. In the present disclosure, aₖ=[0.1, 0.2, 0.5], that is, there are three types of one-dimensional convolution kernels with different sizes. The scale of the k-th type of the time convolution kernel is denoted as [symbol] ^{k} and defined as:

$${𝒜}^{k} = ( 1 , {α}_{k} \cdot L ) \qquad ( 1 )$$

For a given electroencephalogram data xⁱ|ᵢ₌₁ⁿ∈ [symbol] ^{d×L}˜ [symbol] _{S/T}, n is the number of samples of the electroencephalogram data, and d is the number of channels for collecting the electroencephalogram data. First, a plurality of multi-scale one-dimensional time convolution kernels will be used at the same time to learn dynamic time representation of the electroencephalogram data. The representation obtained after the multi-scale time convolution layer is processed by the logarithm of the average pooling square in order to learn the power features of the dynamic time representation of the electroencephalogram signals.

The time convolution output of the k-th type is denoted as X^{(i,k)}∈ [symbol] ^{T}^{ₙ}^{×d×L}^{ₖ}, where Tₙ represents the number of time convolution kernels, and Lₖ represents the feature length of the output feature. X^{(i,k)} can be specifically expressed as:

$${X}^{( i , k )} = {Φ}_{log} ( {ℱ}_{A P} ( {Φ}_{square} ( {ℱ}_{C o n v} ( {x}^{i} , {𝒜}^{k} ) ) ) ) \qquad ( 2 )$$

where [symbol] _{Conv2D}(·) represents the convolution operation, Φ_{square}(·) is a square function; [symbol] _{AP}(·) represents the average pooling operation, and Φ_{log}(·) represents a logarithmic function.

The splicing layer will connect the time convolution kernel outputs of all levels in series in the feature dimension. Therefore, for the input xⁱ|ᵢ₌₁ⁿ∈ [symbol] ^{d×L}, the output X_{cat}ⁱ∈ [symbol] ^{T}^{ₙ}^{×d×ΣL}^{ₖ} of the multi-scale time convolution layer can be calculated as:

$${X}_{cat}^{i} = Γ ( {X}^{( i , 1 )} , … , {X}^{( i , K )} ) \qquad ( 3 )$$

where Γ(·) represents the serial operation along the feature dimension.

Preferably, the fusion layer uses a 1*1 convolution layer as the attention fusion layer to fuse the features learned by different convolution kernels. The number of convolution kernels in the 1*1 convolution layer is set to t. Preferably, Leaky-ReLU is used as the activation function, and the average pooling layer is used to down-sample the learned representation. After batch standardization, the fused representation from different 1*1 convolution kernels will be flattened and become the attribute of each channel node in the graph representation. Therefore, the attention fusion representation X̅ⁱ_{fuse} fuse for each X_{cat}ⁱ can be calculated as:

$${\overline{X}}_{fuse}^{i} = {ℱ}_{bn} ( {ℱ}_{AP} ( {Φ}_{L - ReLU} ( {ℱ}_{fuse} ( {ℱ}_{dropout} ( {ℱ}_{bn} ( {X}_{cat}^{i} ) ) ) ) ) ) \qquad ( 4 )$$

where [symbol] _{bn}(·) is a batch normalization function, [symbol] _{dropout}(·) is a random dropout layer preventing over-fitting, [symbol] _{fuse}(·) is a 1*1 convolution function, Φ_{L-ReLU}(·) is a Leaky-ReLU activation function. X̅ⁱ_{fuse}∈ [symbol] ^{t×C×0.5*Σf}^{ₖ} is reshaped as Xⁱ_{fuse}∈ [symbol] ^{C×t*0.5*Σf}^{ₖ} to construct the attribute of each node (the electroencephalogram signal channel) in the graph representation:

$${X}_{fuse}^{i} = {ℱ}_{reshape} ( {\overline{X}}_{fuse}^{i} ) \qquad ( 5 )$$

The graph convolution module is realized by a graph convolution network using a general messaging framework:

$${X}_{out} = GNN ( A , {X}_{fuse}^{i} ) \qquad ( 6 )$$

where A=Xⁱ_{fuse}Xⁱ_{fuse}^{T}∈ [symbol] ^{d×d}

The attention embedding module obtains the importance of each channel through a similarity calculation, and then carries out a weighted summation based on this. Specifically, first, the global mean representation is calculated:

$${h}_{mean} = \frac{1}{d} {\sum }_{i = 1}^{d} {x}_{out}^{i} \qquad ( 7 )$$

Thereafter, each channel xₒᵤₜⁱ|ᵢ ^{d} and hₘₑₐₙ are subjected to inner product to obtain a similarity representation, which can be regarded as the expression of the importance degree belonging to each channel. Finally, the final embedded vector representation is obtained by weighted summation:

$${h}_{o u t} = softmax ( {h}_{m e a n} {X}_{o u t}^{T} ) {X}_{o u t} \qquad ( 8 )$$

According to the further setting of the present disclosure, in Step 4, a decoupling module for decoupling features and respective classifiers thereof are constructed, and are trained and tested.

Specifically, the adversarial training method is used to realize feature decoupling and classification. First, hₒᵤₜ is decoupled into an identity-related feature had and a task-related feature hₜₐₛₖ by a decoupler [symbol] _{D}(·), and then the classifier [symbol] _{C-id}(·) is trained to realize correct classification, which is iteratively optimized through a cross entropy loss.

$${ℒ}_{c 1} = - {𝔼}_{( {x}_{\text{?}} , {y}_{\text{?}} ) ∼ {\tilde{D}}_{\text{?}}} \sum {y}_{s} log ( {ℱ}_{C - id} ( {h}_{sepc - id} / {h}_{inv - id} ) ) \qquad ( 9 )$$

$${ℒ}_{c 2} = - {𝔼}_{x ∼ {\tilde{D}}_{\text{?}}} \sum {y}_{task} log ( {ℱ}_{C - id} ( {h}_{task} ) ) \qquad ( 10 )$$

$$\text{?} \text{indicates text missing or illegible when filed}$$

Thereafter, the classifiers are fixed, and the two decoupling features of the decouplers are trained to deceive the classifiers of each other. The present disclosure uses a minimized negative entropy to encourage such deception effect:

$${ℒ}_{e n t} = {\sum }_{x ∼ {\tilde{D}}_{\text{?}}} log ( {ℱ}_{C - id} - ( {h}_{task} ) ) + {\sum }_{x ∼ {\overline{D}}_{\text{?}}} log ( {ℱ}_{C - task} ( {h}_{inv - i d} ) ) \qquad ( 11 )$$

$$\text{?} \text{indicates text missing or illegible when filed}$$

The first item indicates deceiving the identity classifier with task features, and the second item indicates deceiving the task classifier with identity features.

The present disclosure realizes the distribution alignment of the source domain and the target domain through adversarial training, and specifically, the training loss function can be expressed as:

$${ℒ}_{d} = - {𝔼}_{x ∼ {\tilde{D}}_{s}} [ log ( {ℱ}_{C - domain} ( {h}_{inv - id} ) ) ] - {𝔼}_{x - {\tilde{D}}_{\text{?}}} [ log ( 1 - {ℱ}_{C - domain} ( {h}_{inv - i d} ) ) ] \qquad ( 12 )$$

$$\text{?} \text{indicates text missing or illegible when filed}$$

Preferably, the present disclosure further decouples the identity information into intra-domain specific identity information and inter-domain invariant identity information, and reduces the difference of prediction results therebetween through L1 norm constraint, specifically, which can be expressed as:

$${L}_{\text{?}} = {‖ {ℱ}_{C - i d} ( {h}_{i n v - i d} ) - {F}_{C - i d} ( {h}_{spec - i d} ) ‖}_{1} \qquad ( 13 )$$

$$\text{?} \text{indicates text missing or illegible when filed}$$

Preferably, a better decoupling effect is obtained by updating the network through the mutual information loss. That is, the mutual information loss between the inter-domain invariant identity feature h_{inv-id} and the paradigm task-related feature hₜₐₛₖ is calculated through the first mutual information network, and the mutual information loss between the inter-domain invariant identity feature h_{inv-id} and the intra-domain specific identity feature h_{sped-id} is calculated through the second mutual information network, which are specifically expressed as:

$${ℳℐ}_{1} ( {h}_{i n v - i d} , {h}_{task} ) = {𝒯}_{1} ( {h}_{inv - id} , {h}_{task} ; θ ) - log ( {e}^{{𝒯}_{1} ( {h}_{inv - id} , {h}_{task}^{′} ; θ )} ) \qquad ( 14 )$$

$${ℳℐ}_{2} ( {h}_{i nv - id} , {h}_{spec - id} ) = {𝒯}_{2} ( {h}_{i nv - id} , {h}_{s p ec - id} ; θ ) - log ( {e}^{{𝒯}_{2} ( {h}_{inv - id} , {h}_{spec - id}^{′} ; θ )} ) \qquad ( 15 )$$

where hₜₐₛₖ and h_{spec-id} represent the edge distributions sampled from hₜₐₛₖ and h_{sped-id}, respectively; [symbol] _{1/2}(·) represent the first mutual information network and the second mutual information network, respectively, that is, the fully connected layer network module; and θ is a learnable parameter.

According to the further setting of the present disclosure, in Step 5, the network model is trained. In order to optimize the network parameters, the present disclosure uses a back propagation method, and updates the network parameters through iteration until the required standard is reached. The training update mode is as shown in the following table 1.

TABLE 1
a working flow chart of a brain fingerprint identification
method with paradigm adaptive decoupling

```
  Input: labelled source domain data [symbol]   _{S} , unlabeled
  target domain data to be predicted [symbol]   _{T} , feature
extractor F , decoupler D , mutual information network MI , classifier C ;
  Output: trained feature extractor \tilde{F} ,
  decoupler \tilde{D} , and classifier \tilde{C} .
  1, extracting features through Formulas (2)-(8);
  2, updating F, D, C through Formulas (9)-(10).
  3, updating F, D by through Formula (11)
  4. updating F, D and C through Formula (12)-(13)
  5, updating D, MI through Formulas (14)-(15).
  Repeat the above Steps 1-5 until convergence.
```

The cross-period brain fingerprint identification method with paradigm adaptive decoupling according to the present disclosure is compared with the classic and recently proposed methods in the field of a brain-computer interface on two cross-period data sets: SEED-V and an electroencephalogram data set collected based on an RSVP protocol under the domain adaptive framework, and the obtained identification precisions (%) are as shown in the following table:

TABLE 1
cross-period identification precisions (%) and F1 scores of SEED-V

| | Time period | | Time period | | Time period | | | |
|---|---|---|---|---|---|---|---|---|
| | 1→Time period 2 | | 1→Time period 3 | | 2→Time period 3 | | average | |
| | | F1 | | F1 | | F1 | | F1 |
| Model | Precision | score | Precision | score | Precision | score | Precision | score |
| Model of the present | 84.23 | 82.14 | 86 | 84.02 | 80.16 | 78.04 | 83.46 | 81.4 |
| disclosure | | | | | | | | |
| EEGnet-domain | 60.14 | 52.34 | 62.98 | 55.87 | 75.49 | 68.76 | 66.2 | 58.99 |
| adaptive | | | | | | | | |
| TScption-domain | 74.26 | 65.51 | 75.16 | 70.75 | 74.09 | 72.06 | 74.5 | 69.44 |
| adaptive | | | | | | | | |
| brainnet-domain | 79.38 | 74.62 | 74.98 | 72.56 | 79.52 | 76.62 | 77.96 | 74.6 |
| adaptive | | | | | | | | |

TABLE 2
cross-period identification precisions (%) and F1 scores of
an electroencephalogram data set based on an RSVP protocol

| | Time period 1→Time period 2 | |
|---|---|---|
| model | Precision | F1 score |
| Model of the present disclosure | 85.7 | 85.7 |
| EEGnet-domain adaptive | 71.37 | 71.18 |
| TScption-domain adaptive | 83.72 | 83.78 |
| brainnet-domain adaptive | 72.8 | 70.46 |

The embodiments of the present disclosure have been described in detail above with reference to the drawing, but the present disclosure is not limited to the described embodiments. It will be obvious to those skilled in the art that many changes, modifications, substitutions and variations can be made to these embodiments, including components, without departing from the principle and spirit of the present disclosure, which still fall within the scope of protection of the present disclosure.

What is claimed is:

1. A cross-period brain fingerprint identification method with paradigm adaptive decoupling, comprising:
Step 1: collecting electroencephalogram data;
Step 2: preprocessing the electroencephalogram data, labeling the electroencephalogram data with identity labels of subjects, and then dividing the electroencephalogram data into a source domain and a target domain according to a sequence of collecting periods, wherein the source domain is a data set with known identity labels of the subjects, and the target domain is a data set with identity labels to be predicted;
Step 3: constructing a feature extraction module with paradigm adaptive decoupling, and training and testing the feature extraction module;
Step 4: constructing a decoupling module for decoupling features and respective classifiers, and training and testing the decoupling module and the classifiers;
wherein the decoupling module for decoupling features comprises an intra-domain specific identity information decoupler, an inter-domain invariant identity information decoupler, a paradigm task information decoupler, a first mutual information network and a second mutual information network;
the intra-domain specific identity information decoupler is configured to decouple a feature representation hₒᵤₜ extracted by the feature extraction module into a domain specific identity feature representation h_{sped-id};
the inter-domain invariant identity information decoupler is configured to decouple the feature representation hₒᵤₜ extracted by the feature extraction module into a domain invariant identity feature representation h_{inv-id};
the paradigm task information decoupler is configured to decouple the feature representation hₒᵤₜ extracted by the feature extraction module into a paradigm task-related feature representation hₜₐₛₖ;
the first mutual information network is configured to calculate a first mutual information loss between the paradigm task-related feature representation hₜₐₛₖ and the domain invariant identity feature representation h_{inv-id}; the second mutual information network is configured to calculate a second mutual information loss between the domain specific identity feature representation h_{sped-id} and the domain invariant identity feature representation h_{inv-id}; and network parameters of the decoupling module are updated through the first mutual information loss and the second mutual information loss;
the classifiers comprise a domain classifier, an identity classifier and a paradigm task classifier;
the domain classifier receives the domain invariant identity feature representation h_{inv-id}, and then performs domain adversarial training to reduce distribution differences between different domains and obtain domain invariant features;
the identity classifier receives the domain specific identity feature representation h_{sped-id} and the domain invariant identity feature representation h_{inv-id} to acquire accurate classification information;
the paradigm task classifier receives the paradigm task-related feature representation hₜₐₛₖ, promotes a decoupling effect, and reduces an influence of interference information on an identity identification task; and
Step 5: using the feature extraction module, the decoupling module and the classifiers which have been trained and verified to realize cross-period brain fingerprint identification.

2. The method according to claim 1, wherein in Step 2, preprocessing the electroencephalogram data comprises: filtering and down-sampling the electroencephalogram data collected in Step 1, and then fragmenting the electroencephalogram data to obtain a plurality of fragments with a sample length of L.

3. The method according to claim 1, wherein in Step 2, dividing the electroencephalogram data into the source domain and the target domain according to the sequence of collecting periods means that: in a time sequence, period data collected first is taken as the source domain with identity labels which is denoted as [symbol] _{S}={X_{S}, Y_{S}}={(x_{S}¹,y_{S}¹), ⋅ ⋅ ⋅ , (x_{S}ⁿ^{_{S}},y_{S}ⁿ^{_{S}})}, n_{S} represents a number of samples in the source domain, x_{S}ⁱ|ᵢ₌₁ⁿ^{_{S}}∈ [symbol] ^{d×L} represents an electroencephalogram data fragment in a period of time, yₛⁱ|ᵢ₌₁ⁿ^{_{S}}∈ [symbol] ^{C} represents an identity label of an i-th subject in the source domain, and C represents a number of the subjects; period data collected later is taken as unlabeled target domain of identities to be predicted, which is denoted as [symbol] _{T}={X_{T}}={x_{T}¹, ⋅ ⋅ ⋅ , x_{T}ⁿ^{ₜ}}, x_{T}ⁱ|ᵢ₌₁ⁿ^{ₜ}∈ [symbol] ^{d×L} represents an electroencephalogram data fragment in a period of time, and nₜ represents a number of samples in the target domain.

4. The method according to claim 1, wherein in Step 3, the feature extraction module with paradigm adaptive decoupling comprises a multi-scale convolution module, a graph convolution module and an attention embedding module;
the multi-scale convolution module comprises a plurality of parallel one-dimensional convolution layers, a splicing layer, a fusion layer and a filtering layer; the plurality of one-dimensional convolution layers have convolution kernels with different sizes; the multi-scale convolution module receives data of the source domain and the target domain, processes the data in multi-time dimension through a plurality of parallel one-dimensional convolution kernels with different sizes, and outputs features of different levels as an input of the splicing layer; the splicing layer splices an output of one-dimensional convolution layers of different levels as an input of the fusion layer; the fusion layer fuses the features learned by different convolution kernels output by the splicing layer, and flattens the features as an input of the filtering layer;
the graph convolution module comprises a plurality of parallel graph convolution networks, and is configured to mine a topological relation and spatial information between channels in a data-driven manner;
the attention embedding module is configured to act on outputs of graph convolution of different levels, and transform a graph structure into an embedding vector through an attention mechanism as an input of decouplers.

5. The method according to claim 1, wherein in a training process, the intra-domain specific identity information decoupler, the inter-domain invariant identity information decoupler and the paradigm task information decoupler decouple hₒᵤₜ into the domain specific identity feature representation h_{sped-id}, the domain invariant identity feature representation h_{inv-id} and the paradigm task-related feature representation hₜₐₛₖ, respectively, and then train the identity classifier [symbol] _{C-id}(·) and the paradigm task classifier [symbol] _{C-task} to realize correct classification, which are iteratively optimized through a cross entropy loss.
$${ℒ}_{c 1} = - {𝔼}_{( {x}_{\text{?}} , {y}_{\text{?}} ) ∼ {\tilde{D}}_{\text{?}}} \sum {y}_{s} log ( {ℱ}_{C - i d} ( {h}_{spec - i d} / {h}_{inv - i d} ) ) \qquad ( 9 )$$
$${ℒ}_{c 2} = - {𝔼}_{x - {\tilde{D}}_{\text{?}}} \sum {y}_{task} log ( {F}_{C - task} ( {h}_{task} ) ) \qquad ( 10 )$$
$$\text{?} \text{indicates text missing or illegible when filed}$$
where [symbol] ₓ_{_{S}}_{,y}_{ₛ}_{)˜\tilde{D}}_{_{S}} represents a feature representation extracted from source domain data; [symbol] _{x˜\tilde{D}}_{_{s/t}} represents a feature representation extracted from source domain data or target domain data; yₛ represents a real identity label of the source domain data; yₜₐₛₖ represents an task label that a subject carries out when the electroencephalogram data is collected;
the domain classifier [symbol] _{C-domain}(·) realizes a distribution alignment of the source domain and the target domain in a domain adversarial manner, specifically, in an identity-related feature subspace, the source domain and the target domain are aligned through domain adversarial training:
$${ℒ}_{d} = - {𝔼}_{x - {\tilde{D}}_{\text{?}}} [ log ( {ℱ}_{C - domain} ( {h}_{inv - id} ) ) ] - {𝔼}_{x - {\tilde{D}}_{\text{?}}} [ log ( 1 - {ℱ}_{C - domain} ( {h}_{i n v - i d} ) ) ] \qquad ( 12 )$$
$$\text{?} \text{indicates text missing or illegible when filed}$$
where [symbol] _{x˜\tilde{D}}_{_{S}}[log( [symbol] _{C-domain}(h_{inv-id})] represents that an encouragement that the domain classifier [symbol] _{C-domain}(·) correctly predicts the source domain data, and [symbol] _{x˜\tilde{D}}_{_{S}}[log(1− [symbol] _{C-domain}(h_{inv-id})] represents an encouragement that features decoupled by the inter-domain invariant identity information decoupler deceive [symbol] _{C-domain}(·) to obtain the domain invariant identity feature.

6. The method according to claim 1, wherein the domain specific identity feature representation h_{sped-id} and the domain invariant identity feature representation h_{inv-id} are constrained by L1 norm.

7. The method according to claim 1, wherein the first mutual information loss [symbol] ₁(h_{inv-id},hₜₐₛₖ) between the domain invariant identity feature representation h_{inv-id} and the paradigm task-related feature representation hₜₐₛₖ is calculated through the first mutual information network, and the second mutual information loss [symbol] ₂(h_{inv-id},h_{spec-id}) between domain invariant identity feature representation h_{inv-id} and the domain specific identity feature representation h_{sped-id} is calculated through the second mutual information network, which are expressed as:
$${ℳℐ}_{1} ( {h}_{inv - i d} , {h}_{task} ) = {𝒯}_{1} ( {h}_{i n v - i d} , {h}_{task} ; θ ) - log ( {e}^{{𝒯}_{1} ( {h}_{inv - id} , {h}_{task}^{′} ; θ )} ) \qquad ( 14 )$$
$${ℳℐ}_{2} ( {h}_{inv - id} , {h}_{spec - i d} ) = {𝒯}_{2} ( {h}_{inv - id} , {h}_{spec - i d} ; θ ) - log ( {e}^{{𝒯}_{2} ( {h}_{inv - id} , {h}_{spec - id}^{′} ; θ )} ) \qquad ( 15 )$$
where hₜₐₛₖ ′ and h_{spec-id}′ represent edge distributions sampled from hₜₐₛₖ and h_{sped-id}, respectively; [symbol] _{1/2}(·) represents the first mutual information network and the second mutual information network, respectively; θ is a learnable parameter.

8. A cross-period brain fingerprint identification system for implementing the method according to any one of claim 1, comprising:
a data collecting module, configured to collect electroencephalogram data;
a data preprocessing module, configured to preprocess the electroencephalogram data;
an identifying module, configured to realize cross-period brain fingerprint identification according to preprocessed electroencephalogram data by using the feature extraction module, the decoupling module and the classifiers which have been trained and verified in advance.

9. A computing device, comprising a memory and a processor, wherein executable codes are stored in the memory, and the processor, when executing the executable codes, implement the method according to any one of claim 1.
